(19) United States
(12) Patent Application Publication
(10) Pub. No.: US 2007/0016043 A1
(43) Pub. Date: Jan. 18, 2007

(54) Bone densitometry system for public use

(75) Inventor: Richard Franklin Morris, Stoughton, WI (US)

Correspondence Address: QUARLES & BRADY LLP, 411 E. WISCONSIN AVENUE, SUITE 2040, MILWAUKEE, WI, 53202-4497, US

(73) Assignee: General Electric Company

(21) Appl. No.: 11/150,550

(22) Filed: Jun. 10, 2005

(51) Int. Cl. A61B 8/00

(52) U.S. Cl. 600/443

(57) ABSTRACT

An ultrasonic bone densitometer system is provided in a kiosk environment using computer driven patient instructions and simplified sanitizing procedures to allow unsupervised use by members of the public for screening purposes.

### STATEMENT REGARDING FEDERALLY SPONSORED RESEARCH OR DEVELOPMENT

--

### CROSS-REFERENCE TO RELATED APPLICATIONS

--

### BACKGROUND OF THE INVENTION

Osteoporosis, or loss of bone mineralization, is becoming increasingly prevalent as the average age of the population increases.

Bone health is currently assessed using one of two principal techniques. The first technique employs a low intensity, dual energy, x-ray beam to distinguish bone from the surrounding soft tissue and provide a reading of areal bone density. Such equipment may provide a table for supporting the patient in a supine position while the patient is scanned with an x-ray beam directed across ends of a C-arm positioned vertically on either side of the patient and moved across the patient. Dual energy x-ray machines normally require a dedicated room and proper supervision by a trained healthcare professional.

A second technique for bone density measurement employs an ultrasonic acoustic signal transmitted across the heel bone or os calcis of the patient's foot. In such a system, the patient places his or her heel in a compact, floor supported unit, having a pair of ultrasonic transducers at the rear of a foot-sized opening. The sound is coupled between the transducers and the heel with a liquid coupling medium such as a gel or alcohol applied to the heel. While this device is somewhat simpler to use than the dual energy densitometer, a trained healthcare professional is still required to instruct a patient in the use of the densitometer, properly position the patient's foot, and to clean the machine between uses.

Considerable public benefit could be obtained by a screening program to find those at high risk for osteoporosis. Public agencies and health institutions are reluctant to undertake such screening operations because of the potentially large costs of screening many individuals. The relative unfamiliarity of the public with densitometers and their inaccessibility further complicates this effort.

### SUMMARY OF THE INVENTION

The present inventor has recognized that large scale screening for severe osteoporosis might be possible using the model of “free” blood pressure checking kiosks found in stores and malls. In this model, the cost of the equipment is offset by advertising revenue or similar third party payments providing the patient with a substantially free service.

Generally, bone densitometers are far more complex and less familiar to the public than blood pressure machines, and ultrasonic bone densitometers in particular currently require careful positioning of the patient's foot and cleaning of the machine between uses. The present invention addresses these issues by providing computer-sequenced instructions to the patient using an eye-level display that also provides feedback guiding the patient in positioning of his or her foot. Spray jets used to automatically apply alcohol to the patient's foot for acoustic coupling also serve to disinfect patient contacting surfaces of the machine. A disposable paper cover may also be used for this purpose.

Specifically then, the present invention provides a public, bone densitometer having a seat and a foot support fixed with respect to the seat and sized to receive and locate a person's foot in a predetermined orientation when the person is seated on the seat. Ultrasonic transducers are positioned in opposition about a heel of the foot as positioned on the support for ultrasonic measurement of the person's os calcis. A display terminal is accessible to the seated person when the person is seated upright and the person's foot is positioned in the foot support so as to be scannable by the ultrasonic transducers. An electronic computer executes a stored program to provide a sequence of instructions on the display instructing the person of the steps of obtaining an ultrasonic bone scan. The computer further measures the ultrasonic characteristics of the person's os calcis and outputs to the person an indication of the measurement.

Thus, it is one object of the invention to provide a bone densitometer that may be used in a public setting without healthcare professionals for free or low cost bone density screening. An eye level display provides instructions accessible to the patient during the measurement process to allow the patient to manage their own bone density scan.

The display terminal may include input keys and the stored program may further accept from the person physiological data about the person selected from the group consisting of gender, age and weight.

It is thus another object of the invention to provide the person with a context to their bone density reading, for example, how their bone density relates to their peers by age, gender, and weight, thereby offering improved guidance to the patient absent a healthcare professional.

The stored program may further check the measurement of the ultrasonic characteristics of the person's os calcis against reasonable ranges for individuals with similar physiological data to output a measure of bone health if the measurement is within reasonable ranges and to not output a measure of bone health otherwise.

Thus, it is another object of the invention to block possibly inaccurate measurements under the assumption that those measurements will not be reviewed by a healthcare professional in the kiosk setting.

The stored program may compare at least two measurements of the ultrasonic characteristics of the person's os calcis before outputting a measure of bone health. Only if the measurements correspond is a reading output, and not otherwise. The measurements, for example, may be speed of sound and broadband ultrasonic attenuation.

Thus, it is another object of the invention to provide additional assurance that the patient is receiving accurate measurements in the absence of an experienced health care professional.

The sequential instructions may include instructions on positioning the foot within the foot positioner.

It is thus one object of the invention to allow the patient to receive instructions on foot positioning while they are actually positioning their foot within the foot support.

The instructions may provide feedback to the person based on a sensing of the position of the foot, for example, through a switch activated by the person's foot or through a sensing of the foot using ultrasonic transducers.

Thus, it is another object of the invention to allow interactive positioning of the foot increasing the likelihood of an accurate measurement without the presence of a trained healthcare professional.

The ultrasonic signals may provide an image of the heel to the person displayed on the display monitor and the image may be a real time image.

It is thus another object of the invention to allow real time interaction between the patient and the machine to provide for correct patient positioning with a variety of patients.

The sequential instructions may include instructions warning the person about the next steps in operation of a public, bone densitometer before the steps are taken.

It is thus another object of the invention to minimize the possibility that the patient is startled, for example, by the application of the alcohol or the inflation of coupling bladders, given the relative unfamiliarity of the public with equipment of this type.

The sequential instructions may include animation showing operation of the bone densitometer.

It is thus another object of the invention to provide for improved tools for patient communication that can easily convey information even to those who may have trouble reading or comprehending written instructions.

The instructions may include instructions related to managing the hygiene of the foot positioner, including the advancing of a paper cover from a paper roll over a portion of the foot support.

It is thus another object of the invention to allow enlisting of the patient to manage hygiene issues associated with such equipment.

The bone densitometer may include sensors on a paper roll and in an alcohol supply providing indication of the supply paper and alcohol in the bone densitometer.

It is thus another object of the invention to allow the consumables of the device to be readily replenished without constant supervision of the device.

These particular objects and advantages may apply to only some embodiments falling within the claims and thus do not define the scope of the invention.

### BRIEF DESCRIPTION OF THE FIGURES

FIG. 1 is a perspective view of a kiosk of the present invention showing a patient seat, a display terminal, and a foot positioner as used by a person obtaining a bone density measurement;

FIG. 2 is top plan view of the foot positioner of FIG. 1 showing the heel and toe portions, the latter covered by a printed paper cover and the former opposed by ultrasonic transducers and alcohol spray jets;

FIG. 3 is a cross-sectional view through the densitometer of FIG. 2 taken along lines 3-3 showing positioning of the paper roll for feeding paper over the toe portion of the foot positioner, an alcohol tank providing alcohol for the nozzles and a heel switch for detecting presence of the patient's foot;

FIG. 5 is a schematic block diagram of the densitometer of FIG. 1 showing a central computer communicating with various components, jets, pumps and reservoirs of the present invention and the terminal used by the patient;

FIG. 6 is a flowchart of the steps of a program executed by the processor of FIG. 5; and

FIG. 7 is a sample display screen of the display of FIG. 1.

### DETAILED DESCRIPTION OF THE PREFERRED EMBODIMENT

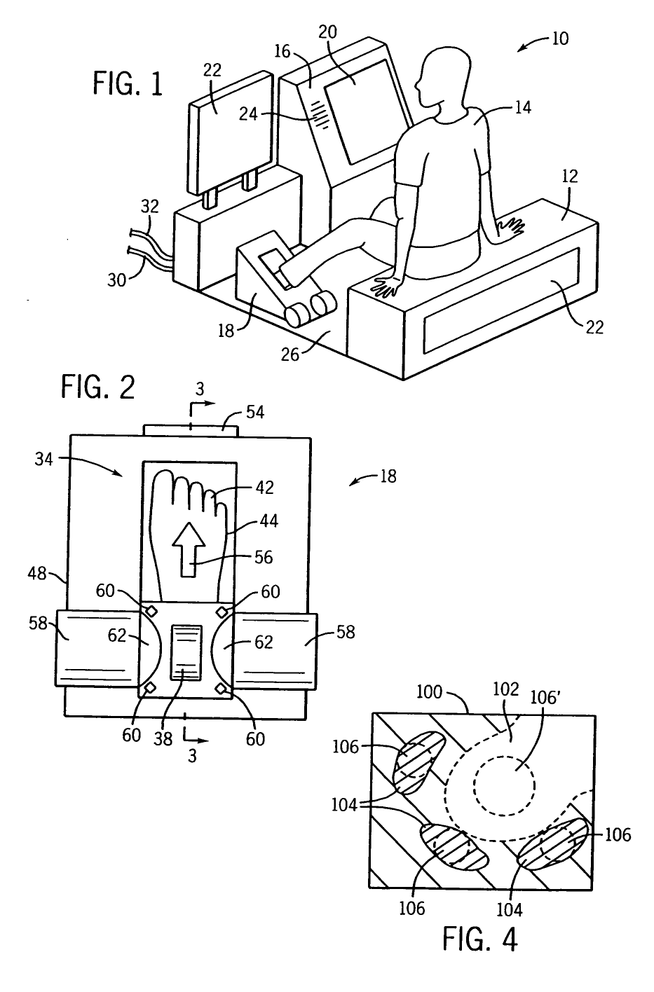

Referring now to FIG. 1, the present invention provides a bone densitometer kiosk 10. In the preferred embodiment, the kiosk 10 includes a bench 12 to support a patient 14 in a seated position, facing a display terminal 16, with one foot received by a floor unit 18.

The display terminal 16 provides a touch screen 20 allowing both display and data entry capabilities and is positioned to be easily viewed and operated by the patient 14 when seated upright on the bench 12 with her foot in the floor unit 18.

The kiosk 10 may include places for advertising placards 22 and advertising may also be periodically displayed on the touch screen 20 or on the bench 12.

The elements of bench 12, the display terminal 16, and floor unit 18 may be attached to a floor unit 26 or may be individually assembled in a store, mall or the like. Electrical connections 30 are provided for electrical power to power the kiosk 10 and optionally an Internet connection 32 that may be used to provide for service calls.

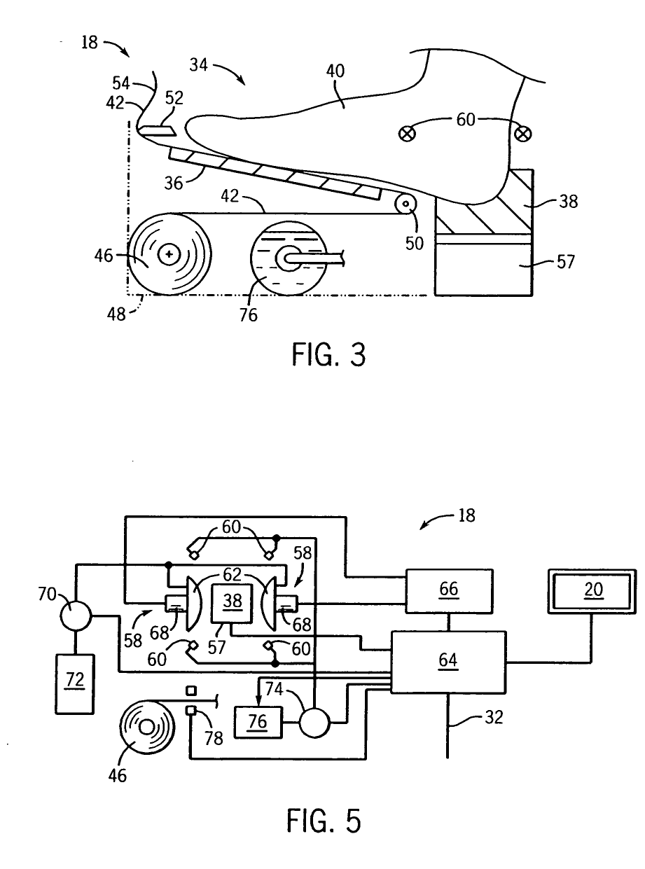

Referring now to FIGS. 2 and 3, the floor unit 18 may include an upwardly opening foot support 34 having a front toe plate 36 and a rear heel block 38 together supporting a person's foot 40 with the toes angled slightly upward in a comfortable attitude for the seated patient 14. The toe plate 36 may be covered with a disposable paper sheet 42 having an outline 44 of the front of the foot so as to guide the patient in placement of the foot on the toe plate 36. The paper sheet 42 may be drawn from a paper roll 46 within a housing 48 of the floor unit 18 unrolling about a horizontal axis beneath the toes of the foot, proceeding toward the heel around an idler roller 50 and reversing direction to pass along the upper surface of the toe plate 36 and to extend from the front surface of the housing 48 past a knife edge 52.

Before use of the floor unit 18, the patient is instructed to grab the extending portion 54 of the paper sheet 42 and to draw a fresh sheet up over the toe plate 36 (in the direction of an arrow 56 printed on the paper sheet 42) to align a new outline 44 of a foot within the area of the foot support 34.

The heel of the foot 40, when the foot is properly positioned, rests on a concave heel block 38 supported on a switch unit 57. When the foot 40 is placed on the foot support 34, the heel of the foot presses down on the switch unit 57 providing an indication that the foot is so positioned.

Referring to FIG. 2, the heel block 38 is located at the rear of the housing 48 toward the patient 14 and between ultrasonic transducer modules 58. The ultrasonic transducer modules 58 are opposed across the heel block 38 so as to pass an ultrasonic beam through the os calcis of the patient's foot. Ultrasonic modules 58 and their operation in a densitometer are generally described in U.S. Patents: U.S. Pat. No. 6,520,914 Ultrasonic densitometer with pre-inflated fluid coupling membranes; U.S. Pat. No. 6,517,487 Ultrasonic densitometer with opposed single transducer and transducer array; U.S. Pat. No. 6,491,635 Digital ultrasonic densitometer; U.S. Pat. No. 6,432,057 Stabilizing acoustic coupler for limb densitometry; U.S. Pat. No. 6,364,837 Contact digital ultrasonic densitometer; U.S. Pat. No. 6,305,060 Method of making a thin film acoustic array; U.S. Pat. No. 6,277,076 Ultrasonic densitometer with pre-inflated fluid coupling membranes; U.S. Pat. No. 6,027,449 Ultrasonometer employing distensible membranes; U.S. Pat. No. 6,012,779 Thin film acoustic array; and U.S. Pat. No. 5,840,029 Imaging ultrasonic densitometer, all assigned to the assignee of the present invention and hereby incorporated by reference.

To the front and rear of each ultrasound module 58 are alcohol jets 60 which may spray the heel block 38 with alcohol before the foot is positioned and which spray the sides of the patient's foot with alcohol after it is positioned, to provide a coupling medium between inflatable elastic bladders 62 forming the operating faces of the ultrasound modules 58. The alcohol spray thus serves both to sanitize the foot contacting surfaces and provide for good acoustic coupling between the foot and the ultrasound modules 58.

Referring now to FIG. 5, the elements of the kiosk 10 are controlled by a microcontroller 64, typically in the floor unit 18, working in conjunction with a digital signal processor 66. The digital signal processor 66 is connected to the ultrasonic transducers 68 of the transducer modules 58 to synthesize ultrasonic waveforms and analyze the same for making of ultrasonic bone densitometry measurements. The digital signal processor 66 may compute, for example, speed of sound (SOS) and broadband ultrasonic attenuation (BUA) that provide an indication alone or in combination of bone density. Preferably these measurements are made at a variety of ultrasonic pathways through the heel, for example, by using a transducer array so as to be able to generate an image or BUA, SOS or attenuation image of sound passing through the heel.

The data obtained from the ultrasonic transducer 68 is passed by the digital signal processor 66 to the microcontroller 64 and may be used for calculating bone health and providing images for automatic and interactive patient positioning as will be described.

Inflation of the bladders 62 may be under control of a pump 70 receiving water from a closed reservoir system 72 and having a pump control signal passing to the microcontroller 64. Likewise, a signal from microcontroller 64 may control a pump 74 communicating with an alcohol reservoir 76 to provide alcohol to the alcohol jets 60.

Microcontroller 64 may also receive a signal from reservoir 76 indicating when alcohol needs to be replenished in the reservoir 76 using a float switch, optical height gage or other similar sensor. A sensor system 78, such as an optical or follower switch sensor, may also detect when paper on paper roll 46 has been depleted, and that signal connected to the microcontroller 64. These signals may be used to indicate to the operator that materials need to be replenished or may be transmitted over an Internet connection 32 for a remote service call.

The microcontroller 64 also communicates with the touch screen 20 to display of text and graphics, including animations, as will be described, and to accept from the patient, through touch screen button entries, data and other inputs.

The signal from the switch unit 57 of the heel block 38 is also received by the microcontroller 64.

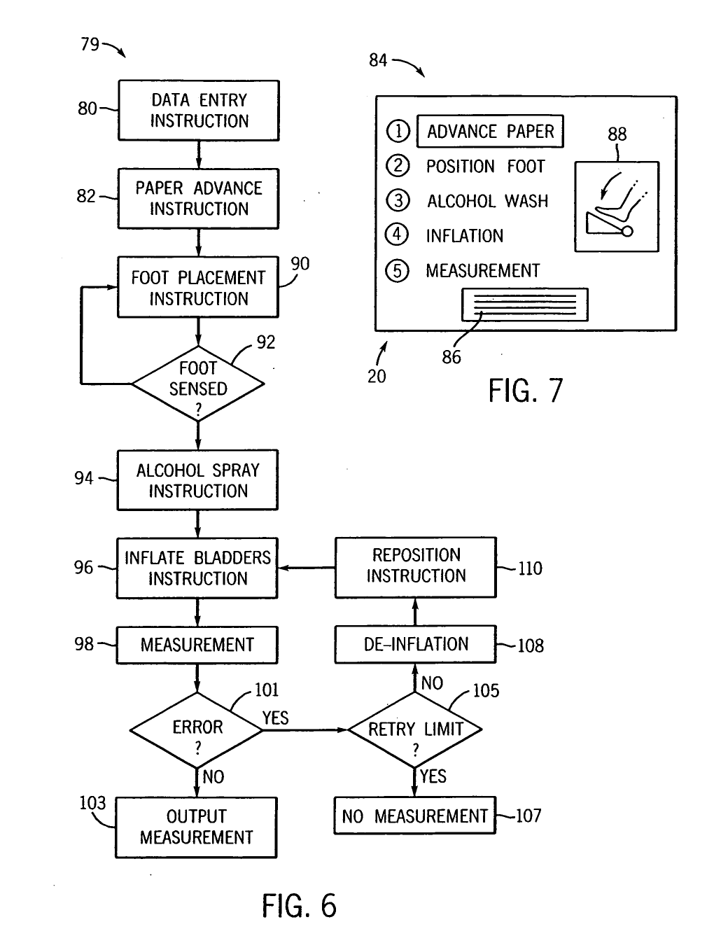

Referring now to FIGS. 1 and 6, the patient approaching the kiosk 10 may get initial instructions as to its purpose from written information 24 on side panels of the display terminal 16 or from information periodically presented on the touch screen 20 under the control of a stored program 79 executed on the microcontroller 64.

Use of the kiosk 10 is initiated by the detection of a pressing of a start button displayed on the touch screen 20 by the program as indicated by program process block 80. When the start button is pressed, the program prompts the patient to enter basic physiological data including patient's age, gender and weight. Some of this physiological data will be used in providing a report to the patient and some of the physiological data will be used as part of an error-checking program to ensure that accurate data is delivered to the patient. The data entry also serves as a threshold for starting the process to distinguish among merely curious bypassers who press the touch screen.

Once data is entered at process block 80, instructions are provided to the patient to begin the measurement process and, in particular, the patient is instructed to advance the paper sheet 42 described above. Once this has been accomplished and confirmed by touch screen entry by the patient, the program activates the alcohol jets 60 to sanitize the heel block 38.

As indicated by FIG. 7, during each of this and the following instructions to the users, the touch screen 20 may display all of the necessary steps in a list 84 with individual steps of the list highlighted as they are performed so as to provide the patient with perspective on the length of the process and the different steps required. Each instruction may be explained in a text box 86 and may have an illustration, for example, an animated illustration in a graphic window 88 showing the patient how to perform the various steps required.

After the paper sheet 42 has been advanced and the heel block 38 sanitized, as illustrated on the touch screen 20, the patient is instructed to position his or her foot within the foot support 34 per process block 90. Decision block 92 then checks the signal from switch unit 57 to determine that the foot is correctly positioned.

Once the foot is in position, the foot is sprayed with alcohol as indicated by process block 94. Prior to the actual activation of the alcohol jets 60, the patient is instructed that the alcohol wash will begin and an animation is shown in graphic window 88 so the patient is not surprised by the experience of the alcohol spray. Optionally, a switch may be pressed by the patient on touch screen 20 to initiate the spray so that they have a sense of control over this process. As the spray is being applied to the patient's heel, the bladders are inflated according to process block 96 to establish good coupling between the foot and transducers.

At process block 98, the microcontroller 64 takes trial ultrasonic measurements of the foot to evaluate whether the foot is correctly positioned. Foot positioning is the most critical element of obtaining a good bone density reading and, accordingly, the adjustment of foot position may be interactive as indicated by the loop of process and decision blocks 101, 105, 108 and 110.

Specifically at decision block 92, the obtained measurements (SOS and BUA) are compared against each other and against the ranges for patients with similar physiological data of age, gender, and weight. Machine analyses of the ultrasound image, according to criteria that will be described below, is also employed to assess whether the measurements are likely to be accurate with the given foot position.

If the measurements correlate reasonably well and the image analyses matches the desired criteria, then at process block 103, the patient is provided with a report typically showing his or her bone health represented as a T score as compared with their peers of the same gender and in a similar age and weight grouping. Optionally, this report may be printed on a self-contained printer within display terminal 16.

If the results of the analyses of decision block 101 are that the measurement likely is in error, the patient is guided in repositioning his or her foot. First, at decision block 105, a retry limit is checked, typically three, placing a limit on the number of times a repositioning will be attempted. If the retry limit is exceeded, the program proceeds to process block 107 and the patient is instructed that no reliable reading could be obtained.

Otherwise at process block 108, the bladders are reduced in pressure to allow the patient to reposition his or her foot according to instructions and guidance provided on the touch screen 20. Referring now to FIG. 4, an ultrasound image 100 of the patient's foot obtained through a concurrent ultrasonic measurement may be displayed to the patient on the touch screen 20. The image 100 will generally show a large bone area 102 bounded by darker peripheral areas 104. Highlighted zones 106 may be superimposed on the image 100 together with instructions, per process block 110, for the patient to manipulate his or her heel so as to align the dark areas 104 with zones 106 corresponding to those areas and the large bone area 102 with the corresponding zone 106′.

Upon a sufficient matching of the areas 102 and 104 and zones 106 and 106′ as determined automatically or upon approval by the patient, the bladders are re-inflated and this measurement process is repeated.

It is specifically intended that the present invention not be limited to the embodiments and illustrations contained herein, but include modified forms of those embodiments including portions of the embodiments and combinations of elements of different embodiments as come within the scope of the following claims.

I Claim:

1. A public, bone densitometer comprising:
a seat;
a foot support fixed with respect to the seat and sized to receive and locate a person's foot in a predetermined orientation when the person is seated on the seat;
ultrasonic transducers positioned in opposition about a heel of the foot as positioned on the support for ultrasonic measurement of the person's os calcis;
a display terminal accessible to a person when the person's foot is positioned in the foot support so as to be scannable by the ultrasonic transducers and the person is seated upright;
an electronic computer executing a stored program to:
(1) provide sequential instructions on the display instructing the person of the steps of obtaining an ultrasonic bone scan;
(2) measure the ultrasonic characteristics of the person's os calcis; and
(3) output to the person an indication of the measurement.

2. The public, bone densitometer of claim 1 wherein the display terminal includes input keys and wherein the stored program further accepts from the person through the input keys, physiological data about the person selected from the group consisting of: gender, age and weight.

3. The public, bone densitometer of claim 2 wherein the stored program further checks the measurement of the ultrasonic characteristics of the person's os calcis against reasonable ranges for individuals with similar physiological data to output a measure of bone health if the measurement is within reasonable ranges and to not output a measure of bone health otherwise.

4. The public, bone densitometer of claim 1 wherein the stored program compares at least two different measurements of the ultrasonic characteristics of the person's os calcis to output a measure of bone health, if the different measurements correspond, and to not output a measure of bone health otherwise.

5. The public, bone densitometer of claim 4 wherein the different measurements are selected from the group consisting of: speed of sound, broadband ultrasonic attenuation.

6. The public, bone densitometer of claim 1 wherein the sequential instructions include instructions on positioning the foot within the foot positioner.

7. The public, bone densitometer of claim 6 wherein the sequential instructions on positioning the foot within the foot positioner provide feedback to the person based on a sensing of the position of the foot.

8. The public, bone densitometer of claim 7 wherein the foot positioner includes a switch activated by the person's foot and wherein the sensing of the position of the foot is a signal from the switch.

9. The public, bone densitometer of claim 7 wherein the sensing of the position of the foot is a signal obtained from ultrasonic transducers.

10. The public, bone densitometer of claim 9 wherein the signal provides an image to the person displayed on the display monitor.

11. The public, bone densitometer of claim 10 wherein the image is a real time image.

12. The public, bone densitometer of claim 1 wherein the sequential instructions include instructions warning the person about the next steps in operation of the public, bone densitometer before the steps are taken.

13. The public, bone densitometer of claim 1 wherein the sequential instructions include animations showing operation of the public, bone densitometer.

14. The public, bone densitometer of claim 1 wherein instructions include instructions relating to managing hygiene of the foot positioner.

15. The public, bone densitometer of claim 1 wherein the instructions include instructions about an application of alcohol to the heel region.

16. The public, bone densitometer of claim 1 wherein the foot support further includes alcohol spray nozzles and wherein instructions include instructions related to an automatic application of an alcohol spray to the heel region.

17. The public, bone densitometer of claim 1 wherein the instructions include instruction related to advancing a paper from a paper roll over a front of the foot support.

18. A bone densitometer comprising:
a foot support sized to receive and locate a human foot in a predetermined orientation;
ultrasonic transducers positioned in opposition about heel of the foot as positioned on the support for ultrasonic measurement of a person's os calcis; and
alcohol spray nozzles positioned near the heel of the foot as positioned on the foot support for applying alcohol to the heel when the foot is so positioned.

19. The bone densitometer of claim 18 further including a paper roll positioned to dispense paper to cover a toe end of the foot support.

20. The bone densitometer of claim 19 further including sensors providing electronic signal indicating a supply of paper and alcohol in the bone densitometer.
